(19) United States
(12) Patent Application Publication
(10) Pub. No.: US 2014/0213950 A1
(43) Pub. Date: Jul. 31, 2014

(54) BELT FOR RELIEVING BACK PAIN

(71) Applicants: Sang-ho Hwang, Busan (KR); Soo-min Hwang, Busan (KR); Dong-woo Hwang, Busan (KR)

(72) Inventors: Sang-ho Hwang, Busan (KR); Soo-min Hwang, Busan (KR); Dong-woo Hwang, Busan (KR)

(21) Appl. No.: 14/153,341

(22) Filed: Jan. 13, 2014

(30) Foreign Application Priority Data
Jan. 29, 2013 (KR) 10-2013-0009927

(51) Int. Cl. A61F 5/02

(52) U.S. Cl. CPC A61F 5/026
USPC 602/19

(57) ABSTRACT

Disclosed herein is a belt for relieving back pain. The belt includes a belt part which is wrapped around a waist of a user, snap buttons which are provided on an inner circumferential surface of the belt part at positions spaced apart, and a support rod coupled to at least one selected from among the snap buttons in such a way that the support rod is oriented perpendicular to the belt part. The support rod includes a cylindrical body that has on an outer circumferential surface thereof snap buttons, and a filler charged into the cylindrical body. The snap buttons of the support rod include snap buttons which are arranged in a longitudinal direction on the cylindrical body so that the support rod can also be horizontally fastened to the belt part.

### REFERENCE TO RELATED APPLICATIONS

This application claims the priority benefit of Korean Patent Application No. 10-2013-0009927 filed on Jan. 29, 2013, the entire contents of which are incorporated herein by reference.

### FIELD OF THE INVENTION

The present invention relates generally to belts for relieving back pain and, more particularly, to a back-pain relieving belt which can mitigate back pain.

### BACKGROUND OF THE INVENTION

Back pain is a common disease of modern people and is caused by a variety of reasons.

Abnormality of any one among the spine including the cervical spine, the thoracic vertebrae, the lumbar spine, the sacral vertebrae and the caudal spine, the several ligaments and peripheral muscles that surround the spine, the spinal cord and structures pertaining to the spinal nerves can cause back pain. Pain occurs at various locations with different kinds of symptoms.

To prevent such back pain, the stability of the spine, that is, the backbone, is important, and the importance of the stability of the ligaments and muscles that surround the spine is also recently emphasized. If the ligaments and muscles that surround the spine are sufficiently strong, even if deformation of the spine is comparatively severe, pain can be mitigated.

Representative examples of belts for preventing or relieving such back pain or related conventional techniques were proposed in Korean Patent Application No. 10-2006-0068246, No. 10-2011-0087963, etc.

### SUMMARY OF THE INVENTION

Accordingly, the present invention has been made keeping in mind the above problems occurring in the prior art, and an object of the present invention is to provide a belt for relieving back pain which is configured such that a support rod directly strongly presses aching portions of a user and is able to appropriately support several portions of the user that come into contact with the support rod oriented in the vertical direction.

In order to accomplish the above object, the present invention provides a belt for relieving back pain, including a belt part wrapped around a waist of a user, snap buttons provided on an inner circumferential surface of the belt part at positions spaced apart, and a support rod coupled to at least one selected from among the snap buttons in such a way that the support rod is oriented perpendicular to the belt part, the support rod including a cylindrical body having on an outer circumferential surface thereof snap buttons, and a filler charged into the cylindrical body, the snap buttons of the support rod including snap buttons arranged in a longitudinal direction on the cylindrical body so that the support rod is horizontally fastened to the belt part.

The cylindrical body may be made of leather material, and the filler may be made of fabric material.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a view illustrating a belt for relieving back pain according to a preferred embodiment of the present invention; and

FIG. 2 is a view showing usage of the belt according to the embodiment of the present invention.

### DETAILED DESCRIPTION OF THE INVENTION

Hereinafter, a preferred embodiment of the present invention will be described in detail with reference to the attached drawings.

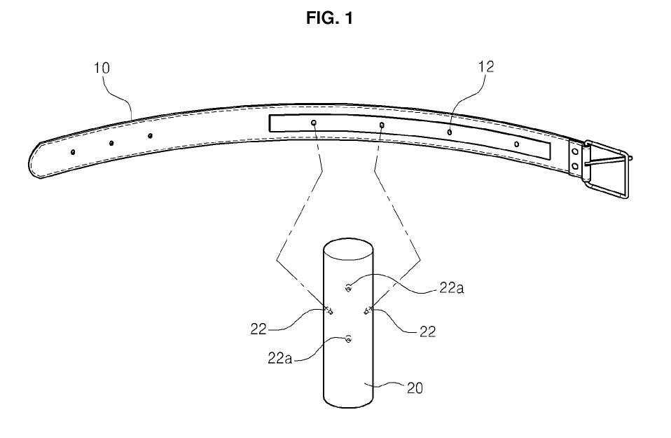

FIG. 1 is a view illustrating a belt for relieving back pain according to a preferred embodiment of the present invention.

The back-pain relieving belt according to the present invention includes a belt part 10 which is wrapped around the waist of a user, snap buttons 12 which are provided on an inner circumferential surface of the belt part 10 at positions spaced apart from each other at regular intervals, and a support rod 20 which is coupled to at least one selected from among the snap buttons 12 in such a way that the support rod 20 is oriented perpendicular to the belt part 10.

The belt part 10 has a similar structure to that of a conventional waist protection belt. Preferably, a leather belt that has a width ranging from 10 cm to 15 cm is used as the belt part 10 to provide reliable support force.

The support rod 20 includes a cylindrical body which has on an outer circumferential surface thereof, snap buttons 22 that correspond to the snap buttons 12 provided on the belt part 10, and a filler which is charged into the cylindrical body.

That is, if female snap buttons are provided on the inner circumferential surface of the belt part 10, male snap buttons are provided on the outer circumferential surface of the support rod 20.

Particularly, it is preferable that the cylindrical body be made of leather material and the filler be made of fabric material.

Furthermore, snap buttons 22a are arranged in the longitudinal direction on the cylindrical body so that the support rod 20 can also be horizontally fastened to the belt part 10.

An example of usage of the back-pain relieving belt according to the present invention will be described herein below.

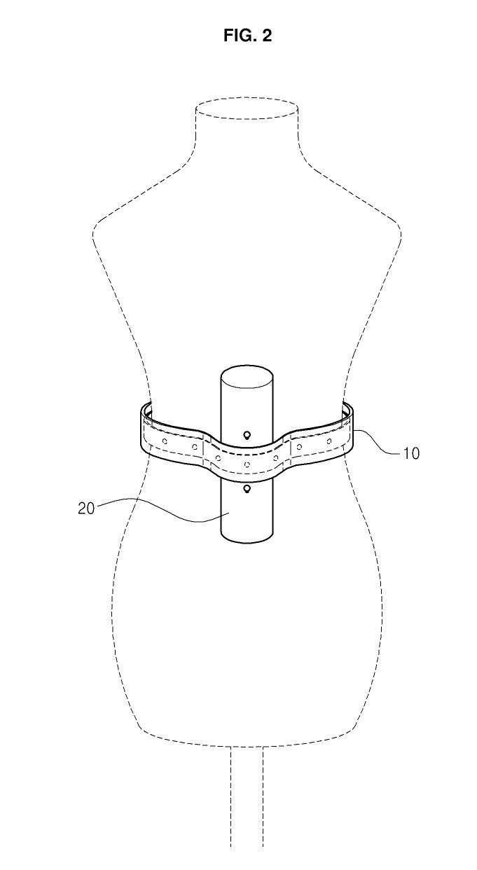

FIG. 2 is a view showing usage of the belt according to the embodiment of the present invention.

As shown in FIG. 2, the support rod 20 is perpendicularly fastened to the belt part 10 in such a way that the snap buttons 22 of the support rod 20 are coupled to selected snap buttons 12 of the belt part 10. Thereafter, the belt part 10 is wrapped around the waist of the user and is fastened.

Then, the support rod 20 that is perpendicularly coupled to the belt part 10 and is vertically oriented presses the waist of the user so that back pain of the user can be relieved.

That is, the support rod 20 that is vertically oriented applies sufficient pressure to the spine of the user and to a depressed portion between costal bones so as to relieve pain. Further, because the support rod that is comparatively long vertically supports the back of the user, pain which occurs in several areas of the spine that are close to the support rod can be effectively relieved.

The support rod is filled with the filler that is made of density-enhanced fabric material so that the satisfactory pressurizing effect is provided but it is prevented from being excessively hard.

In another embodiment, in lieu of the snap buttons, Velcro tape may be used in the same manner.

As described above, a belt for relieving back pain according to the present invention is configured such that a support rod coupled to a belt part by means of snap buttons is vertically or horizontally directly fastened to an aching portion of a user. Thus, the user can easily wear the belt. Therefore, when needed, the belt can be easily used to rapidly relieve back pain.

In addition, the support rod can be easily attached to the belt part or removed therefrom using the snap buttons so that the user can conveniently use the belt.

Although a belt for relieving back pain according to the preferred embodiment of the present invention has been disclosed for illustrative purposes, those skilled in the art will appreciate that various modifications, additions and substitutions are possible, without departing from the scope and spirit of the invention as disclosed in the accompanying claims.

What is claimed is:

1. A belt for relieving back pain, comprising:
a belt part wrapped around a waist of a user;
snap buttons provided on an inner circumferential surface of the belt part at positions spaced apart; and
a support rod coupled to at least one selected from among the snap buttons in such a way that the support rod is oriented perpendicular to the belt part,
the support rod comprising: a cylindrical body having on an outer circumferential surface thereof snap buttons; and a filler charged into the cylindrical body, the snap buttons of the support rod including snap buttons arranged in a longitudinal direction on the cylindrical body so that the support rod is horizontally fastened to the belt part.

2. The belt as set forth in claim 1, wherein the cylindrical body is made of leather material, and the filler is made of fabric material.
